(19) United States
(12) Patent Application Publication
(10) Pub. No.: US 2020/0297447 A1
(43) Pub. Date: Sep. 24, 2020

(54) A DEVICE FOR SENSING THE POSE AND MOTION OF A HUMAN'S ARM-HAND

(71) Applicant: Council of Scientific & Industrial Research, New Delhi (IN)

(72) Inventors: Sanjeev SONI, Chandigarh (IN); Sanjeev VERMA, Chandigarh (IN); Dinesh PANKAJ, Chandigarh (IN); Amod KUMAR, Chandigarh (IN)

(21) Appl. No.: 16/086,160

(22) PCT Filed: Mar. 20, 2017
(86) PCT No.: PCT/IN2017/050101

§ 371 Date: Sep. 18, 2018

(30) Foreign Application Priority Data
Mar. 18, 2016 (IN) 201611009446

(51) Int. Cl. A61B 34/00; A61B 34/37; B25J 13/02

(52) U.S. Cl. CPC A61B 34/74; A61B 34/37; A61B 2017/00207; A61B 2034/742; B25J 13/02

(57) ABSTRACT

A device (100) for measuring pose and/or motion of an operator's hand (1) comprising: a first link (10) being adapted to be fixedly held and the distal end (12) being coupled to a second link (20) so as to allow a rotation motion there between about a first axis (15); the second link (20) being coupled to a third link (30) so as to allow a rotation motion there between about a second axis (25), the second axis (25) being substantially perpendicular to the first axis (15); the first edge (41) of the parallelogram linkage (40) being coupled to the third link (30); the second edge (42) of the parallelogram linkage (40) being coupled to a fourth link (50); the fourth link (50) being coupled to a fifth link (60), a distal end (62) thereof being coupled to a sixth link (70); and a grasper (80) being operably coupled to the sixth link (70).

### FIELD OF THE INVENTION

The present invention relates to a device for sensing the pose & motion of human's hand. The present invention particularly relates to a master device for controlling or assessment or measurement for a variety of applications. The device can be used for medical applications like robotic-surgery etc., assessment & rehabilitation of patients (with upper limb movement disorders) as well as upper limb amputees and for other applications involving skilled/professional hand-arm movements of a human etc.

### BACKGROUND OF THE INVENTION

The state of the art relevant to the present invention contains few of the electro-mechanically controlled devices for sensing the hand motion meant for specific applications. The available devices in this area are complex, bulky and energy intensive, costly. These require training of the operator and are not versatile for use in a variety of applications in the marketplace.

By way of example, U.S. Pat. No. 4,726,248 dated Feb. 23, 1988 teaches a master manipulator of a master-slave type manipulator. The manipulator is used to perform a task (by the operator) in a work-area which is not directly accessible to the operator due to dangerous environment or other conditions. The device comprise of a complicated & bulky/complex linkage system meant for large scale movements. The motion sensors are bulky shafts with motors, gears along with sensors. The manipulator is meant to replicate the wrist motions only. Also, a number of motion transmission elements are involved to sense a single motion. There is no measurement of upper arm motion as well as no gripper provision to grasp/release an object.

By way of example, U.S. Patent Publication No. US 2011/0118752 A1 dated May 19, 2011 teaches a tracking location of a part of human hand. It is based on computer vision method. The sensors are mounted on human hand itself. This means there is a skilled job involved to mount the sensors suitably on hands specific to a person. There can be detachment of sensors/discomfort to the operator. Again, here only wrist motions are tracked. There may be ambiguity in hand gestures while recording through visual based methods. Also the hand gesture pose recognition process needs to be trained using a training database. The sensors used for tracking may not be visual for all the hand postures i.e. might be operator or self-occluded. Pose reconstruction accuracy relies heavily on the camera calibration.

By way of example, U.S. Patent Publication No. US 2009/0132088 A1 dated May 21, 2009 discloses a method for teaching a master expert machine by a skilled worker who transfers his/her professional knowledge in form of elementary motions and subdivided tasks. The human wears 1-2 3D gloves equipped with sensors to record the movements. These sensors record the movements and transfer the data to a slave robot etc. This invention is mainly for the tasks involving multi-fingers. Also, this motion sensing is restricted to palm and 2-3 fingers only.

By way of example, U.S. Pat. No. 9,050,727 B2 dated Jun. 9, 2015 describes master operation input device meant for robotic surgery. The device needs two linear/prismatic linkages to detect the arm translation motion and there is no parallelogram for better rigidity. The links are of longer size and overall bulky. This method requires suitable modifications on the control device to use it effectively. Also, there is no provision for the finger grasper hooks, which can reduce the stability of operation. The inertia of the linkages seems high and its effect on the ease of operation seems prominent.

The drawback of the above (prior) inventions/devices is that they are bulky, expensive and are energy intensive i.e. they need additional motors for gravity balancing (to reduce the load on the operator's hand during the operation). Such a load can modify the natural motion of a human's hand-arm. Additionally it is required to make them modular, light weight, economically viable, easily operable, with desired measurement resolution. Also there is need to have a versatile single device which can be used a variety of discussed applications.

### OBJECTIVES OF THE INVENTION

The main object of the invention is to provide a device for sensing/measurement of the pose & motion of a human's hand-arm which obviates the above drawbacks.

### SUMMARY OF THE INVENTION

This summary is provided to introduce a selection of concepts in a simplified format that are further described in the detailed description of the invention. This summary is neither intended to identify key or essential-inventive concepts of the invention, nor is it intended for determining the scope of the invention.

Accordingly, the present invention provides a device for measuring pose and/or motion of an operator's hand, the device comprising: a first link having a proximal end and a distal end, the proximal end being adapted to be fixedly held and the distal end being coupled to a proximal end of a second link so as to allow a rotation motion there between about a first axis; a distal end of the second link being coupled to a proximal end of a third link so as to allow a rotation motion there between about a second axis, the second axis being substantially perpendicular to the first axis; a parallelogram linkage defining a first edge and a diametrically opposite second edge, the first edge of the parallelogram linkage being coupled to a distal end of the third link; the second edge of the parallelogram linkage being coupled to a proximal end of a fourth link; a distal end of the fourth link being coupled to a proximal end of a fifth link, a distal end thereof being further coupled to a proximal end of a sixth link; and a grasper being operably coupled to a distal end of the sixth link.

In an embodiment of the present invention the device movement is actuated (without any external motors) just due to the natural motion of the hand-arm.

In other embodiment of the present invention the opening/closing of the grasper and other joints of the device are actuated naturally due to human's hand motion to implement energy saving.

In other embodiment of the present invention the device has a modular design so that the desired linkages/parts can be attached in a desired configuration and can be used for a specific application.

In another embodiment, both the upper arm & forearm motions as well as pose can be measured through the single device.

In another embodiment of the present invention, various linkages are attached to each other through revolute joints and are mounted with suitable sensors (encoders) to provide measurement of position and orientation of each joint.

In yet another embodiment of the present invention, the open/close of the grasper are implemented to allow a person to release/grasp an object say a surgical tool, brush, pen etc in a virtual environment.

In yet another embodiment of the present invention, an in built electronic scaling down feature is incorporated in the device such that it helps in reducing the vibration effect in measurements.

In still another embodiment of the present invention, apart from the hand-arm motion, the rotation of human body can also be measured to get complete pose of a human. So, that this device is applicable for measuring the motion of a human performing a task in sitting as well as standing configuration.

In still another embodiment of the present invention a low inertia of the each joint is implemented to have real time motion sensing and thus real-time control of an application based on this data.

In still another embodiment of the present invention, the device is kept light weight and sits on the sides of human during the operation. So that there is no loading or obstruction of human hand motion and thus the natural motion can be recorded without external influence.

In still another embodiment of the present invention, a torsion spring is incorporated in the device to minimize the effect of inertia on human's hand to ease the manipulation of the device.

In still another embodiment of the present invention, a real time electronic interface measures the joint rotations and kinematic algorithm is implemented to get the pose & motion of the hand.

In still another embodiment of the present invention Inertia of each joint of the mechanism is kept minimal such that it minimizes the energy dissipation of human's hand while operating the device.

These and other aspects as well as advantages will be more clearly understood from the following detailed description taken in conjunction with the accompanying drawings and claims.

### BRIEF DESCRIPTION OF THE DRAWING

To further clarify advantages and aspects of the invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings, which are listed below for quick reference.

FIG. 1 illustrates a schematic of the present invention/device.

FIG. 2 is a schematic showing a typical operation of the device.

FIG. 3 is a flow chart of the process as implemented by the processing device; and

FIG. 4 illustrates a block diagram of various sub-modules as may be present within the processing device.

It may be noted that to the extent possible, like reference numerals have been used to represent like elements in the drawings. Further, those of ordinary skill in the art will appreciate that elements in the drawings are illustrated for simplicity and may not have been necessarily drawn to scale. For example, the dimensions of some of the elements in the drawings may be exaggerated relative to other elements to help to improve understanding of aspects of the invention. Furthermore, the one or more elements may have been represented in the drawings by conventional symbols, and the drawings may show only those specific details that are pertinent to understanding the embodiments of the invention so as not to obscure the drawings with details that will be readily apparent to those of ordinary skill in the art having benefit of the description herein.

### DETAILED DESCRIPTION

It should be understood at the outset that although illustrative implementations of the embodiments of the present disclosure are illustrated below, the present invention may be implemented using any number of techniques, whether currently known or in existence. The present disclosure should in no way be limited to the illustrative implementations, drawings, and techniques illustrated below, including the exemplary design and implementation illustrated and described herein, but may be modified within the scope of the appended claims along with their full scope of equivalents.

The term “some” as used herein is defined as “none, or one, or more than one, or all.” Accordingly, the terms “none,” “one,” “more than one,” “more than one, but not all” or “all” would all fall under the definition of “some.” The term “some embodiments” may refer to no embodiments or to one embodiment or to several embodiments or to all embodiments. Accordingly, the term “some embodiments” is defined as meaning “no embodiment, or one embodiment, or more than one embodiment, or all embodiments.”

The terminology and structure employed herein is for describing, teaching and illuminating some embodiments and their specific features and elements and does not limit, restrict or reduce the spirit and scope of the claims or their equivalents.

More specifically, any terms used herein such as but not limited to “includes,” “comprises,” “has,” “consists,” and grammatical variants thereof do NOT specify an exact limitation or restriction and certainly do NOT exclude the possible addition of one or more features or elements, unless otherwise stated, and furthermore must NOT be taken to exclude the possible removal of one or more of the listed features and elements, unless otherwise stated with the limiting language “MUST comprise” or “NEEDS TO include.”

Whether or not a certain feature or element was limited to being used only once, either way it may still be referred to as “one or more features” or “one or more elements” or “at least one feature” or “at least one element.” Furthermore, the use of the terms “one or more” or “at least one” feature or element do NOT preclude there being none of that feature or element, unless otherwise specified by limiting language such as “there NEEDS to be one or more . . . ” or “one or more element is REQUIRED.”

Unless otherwise defined, all terms, and especially any technical and/or scientific terms, used herein may be taken to have the same meaning as commonly understood by one having an ordinary skill in the art.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfil the requirements of uniqueness, utility and non-obviousness.

Use of the phrases and/or terms such as but not limited to “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or variants thereof do NOT necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

The present invention relates to the development of a device which can be used to measure the pose & motion of a human's hand while performing a specific task. This measurement can be used to control a slave manipulator (in case of tele/robotic surgery) or for training through virtual surgery (to mimic a skilled surgeon's movements) or for data generation for a skilled task or for rehabilitation assessment of an upper limb amputee/motor deficient person etc. The measured data will also be useful in devising future autonomous surgical robots or skilled artists etc. Additionally, the invention can be used to evaluate/assess/rehabilitate the hand-arm coordination of say a patient (with upper limb movement disorder) or an upper limb amputee etc.

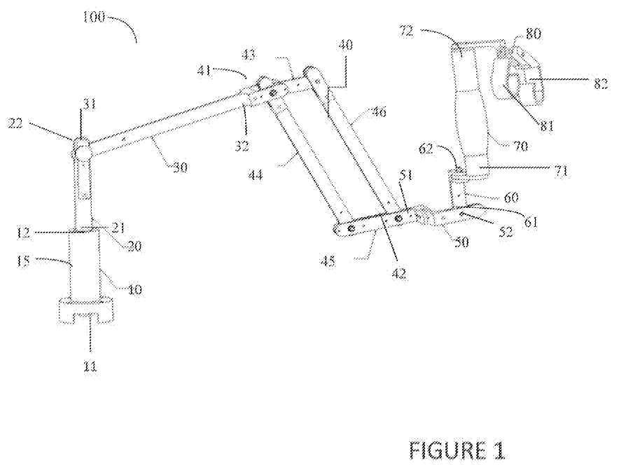

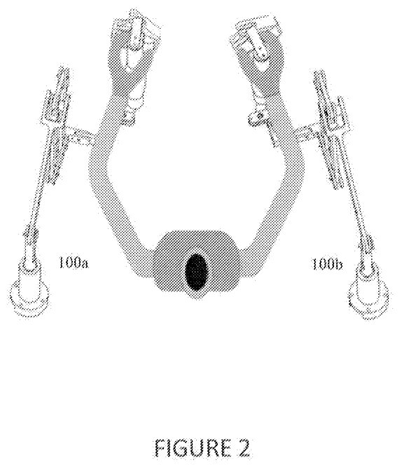

Referring to FIG. 1, there is illustrated a device (100) for measuring pose and/or motion of an operator's hand. As illustrated in FIG. 2, two such devices (100a, 100b) may be placed on either side of the operator for operation thereof. Referring back to FIG. 1, the device (100) comprises a first link (10) having a proximal end (11) and a distal end (12). The proximal end (11) being adapted to be fixedly held and the distal end (12) being coupled to a proximal end (21) of a second link (20) so as to allow a rotation motion there between about a first axis (15).

A distal end (22) of the second link (20) is coupled to a proximal end (31) of a third link (30) so as to allow a rotation motion there between about a second axis (25), the second axis (25) being substantially perpendicular to the first axis (15).

The device (100) comprises a parallelogram linkage (40) defining a first edge (41) and a diametrically opposite second edge (42), the first edge (41) of the parallelogram linkage (40) being coupled to a distal end (32) of the third link (30) and the second edge (42) of the parallelogram linkage (40) being coupled to a proximal end (51) of a fourth link (50).

A distal end (52) of the fourth link (50) being coupled to a proximal end (61) of a fifth link (60), a distal end (62) thereof being further coupled to a proximal end (71) of a sixth link (70); and a grasper (80) being operably coupled to a distal end (72) of the sixth link (70).

In an embodiment of the invention, the parallelogram linkage (40) comprises a seventh link (43), an eighth link (44), a ninth link (45) and a tenth link (46) connected to each other through revolute joints. In an embodiment of the invention, the seventh link (43) and the ninth link (45) are parallel to each other and in an operating state are located parallel to a forearm of the operator and the eighth link (44). In an embodiment of the invention, the eighth link (44) and tenth link (46) are parallel to each other and in an operating state are located parallel to an upper arm of the operator.

By way of a non-limiting example, the fourth link (50), the fifth link (60) and the sixth link (70) in operation are adapted to mimic an operator's wrist movement. In an embodiment of the invention, the grasper (80) defines a first element (81) adapted to contact a thumb of an operator and a second element (82) adapted to be contact at least one of the remaining fingers of the operator.

In an embodiment of the invention, the device (100) further comprises a processing device (400). The processing device (400) in one preferred embodiment is operably coupled to at least one sensor via one or more data acquisition hardware units.

The device (100) may be provided with at least one sensor. Because of the complexity in showing the sensor, the sensors are not illustrated in FIG. 1. However, by way of some non-limiting examples, the device (100) may be provided with at least one of:
a sensor disposed at a location of coupling of the first link (10) and the second link (20) (joint between the first link and the second link);
a sensor disposed at a location of coupling of the second link (20) and the third link (30) (joint between the second link and the third link);
sensor disposed at a location of coupling of the third link (30) and the parallelogram linkage (40) (joint between the third link and the parallelogram linkage);
a sensor disposed at a location of coupling of the parallelogram linkage (40) and the fourth link (50) (joint between the parallelogram linkage and the fourth link);
a sensor disposed at a location of coupling of the fourth link (50) and the fifth link (60) (joint between the fourth link and the fifth link);
a sensor disposed at a location of coupling of fifth link (60) and the sixth link (70) (joint between the fifth link and the sixth link);
a sensor disposed at a location of coupling of the sixth link (70) and the grasper (80) (joint between the sixth link and the grasper);
a sensor disposed at a location of coupling of the seventh link (43) and the eighth link (44) (joint between the seventh link and the eighth link);
a sensor disposed at a location of coupling of the eighth link (44) and the ninth link (45) (joint between the eighth link and the ninth link);
a sensor disposed at a location of coupling of the ninth link (45) and the tenth link (46) (joint between the ninth link and the tenth link);
a sensor disposed at a location of coupling of the tenth link (46) and the seventh link (43) (joint between the tenth link and the seventh link);
a sensor disposed at about the first element (81); and
a sensor disposed at about the second element (82).

Although not illustrated in FIG. 1, the device (100) may further comprise at least one torsion spring for minimizing effect of gravity/inertia on human's hand while operating the device (100).

Likewise, although not specifically illustrated in FIG. 1, the device (100) has at least one of:
the first link (10) having an adjustable length;
the second link (20) having an adjustable length;
the third link (30) having an adjustable length;
the parallelogram linkage (40) having an adjustable size;
the fourth link (50) having an adjustable length;
the fifth link (60) having an adjustable length; and
the sixth link (70) having an adjustable length.

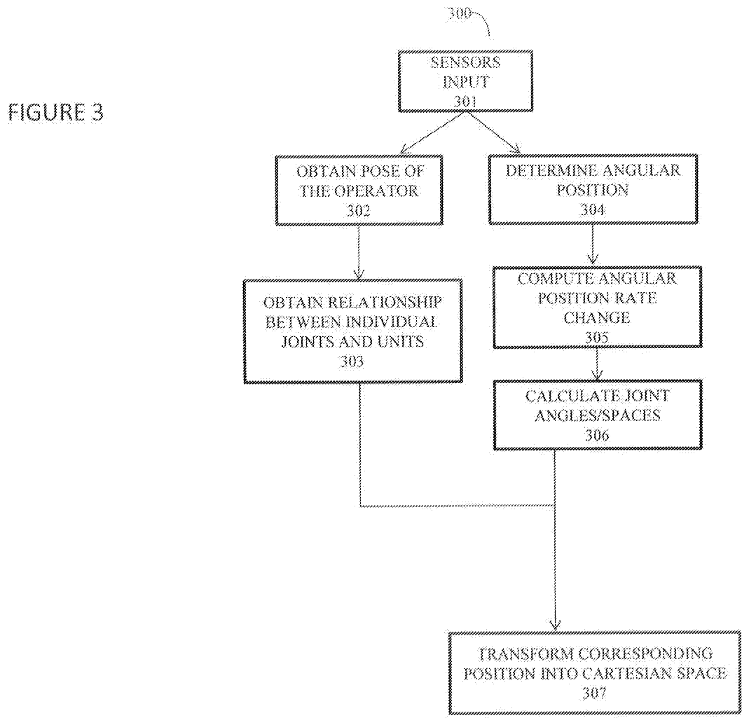

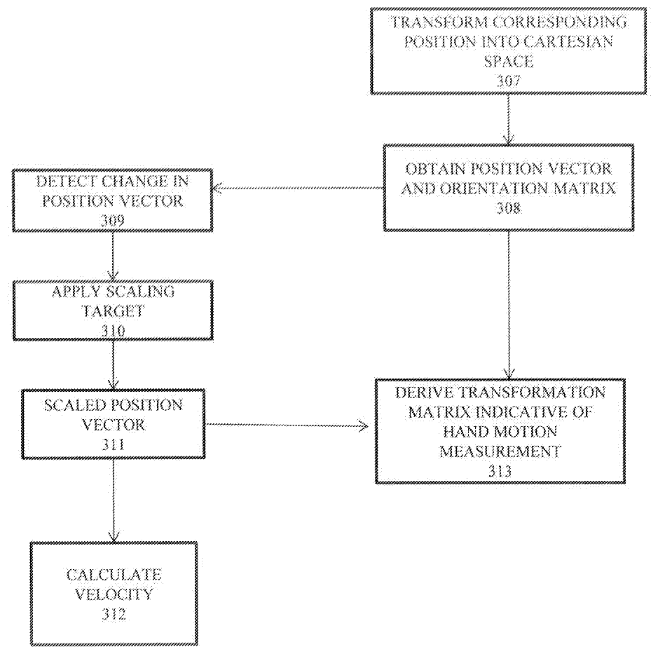

By way of a non-limiting example, and referring to FIG. 3, the processing device may be adapted to a process (300) that comprises:
Obtain (301) input from the at least one sensor via the at least one data acquisition hardware;
Determine (302) a pose of the hand of the operator based on the sensor's input and obtain (303) information pertaining to a relationship between individual joint and the corresponding link;
Determine (304) angular position of a joint corresponding to the sensor's input;
Compute (305) a rate of change of each angular position;
Calculate (306) joint angles or joint spaces on basis of rate of change of angular positions and information pertaining to the device's (100) limited range of motion;
Transform (307) the joint angles or joint spaces into corresponding position in Cartesian space on the basis of the information pertaining to the relationship between individual joint and the corresponding link;
Obtain (308) position vector and orientation matrix;
Detect (309) a change in position vector;
Apply (310) a scaling ratio to obtained scaled position vectors (311);
Derive (312) a velocity on basis of the scaled position vectors thus obtained; and
Derive (313) a transformation matrix indicative of hand motion measurements on basis of the orientation matrix and the scaled position vectors thus obtained.

In an embodiment of the invention, the step of determining a pose of the hand of the operator includes processing sensor inputs pertaining to yaw motion, pitch motion and roll motion of the fourth link (50), the fifth link (60), the sixth link (70) and the grasper (80).

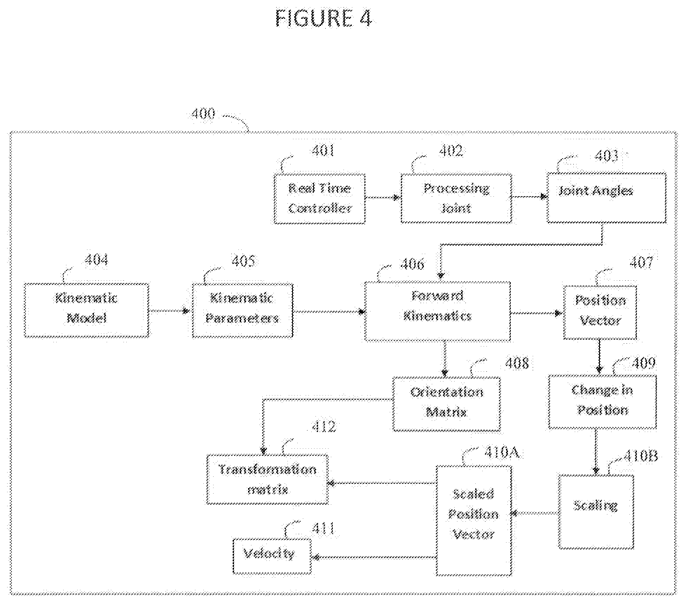

The processing deice (400) may comprise one or more modules for performing the process (300) as described above. By way of a non-limiting example, as shown in FIG. 4, the processing device (400) may comprise a high speed, high performance real time processor (401) that reads angular position of a joint corresponding to the sensor's input and computes the rate at which these angular positions are continuously changing.

The processing device (400) may comprise a module for processing joints (402) and a module for joint angle calculation (403) for calculating joint angles or joint spaces on basis of rate of change of angular positions and information pertaining to the device's (100) limited range of motion. By way of non-limiting example, the limited/restricted range of motion may depend on the work space of the device (100).

The processing device (400) may comprise a kinematic model implementing module (404) and a kinematic parameter determining module (405) adapted to determine a pose of the hand of the operator based on the sensor's input and obtain information pertaining to a relationship between individual joint and the corresponding link.

The processing device (400) may further comprise a forward kinematics algorithm implementing module (406) that transforms joint spaces into corresponding positions in Cartesian space according to the relationship between the individual joints & links defined by the kinematic parameter determining module (405).

Subsequent thereof, position vector and orientation matrix are obtained from forward by the position vector determining module (407) and the orientation matrix calculation module (408), respectively. The position vector is further processed by a change detection module (409) to get a change in position vector.

A scaling factor application module (410A) can apply a scaling factor to change in the position vector (alternatively called as “scaled position vector”) to achieve smooth and precise motion of the ‘devices to be controlled’ with the help of present invention. Scaling (410B) will also help to reduce any sort of vibration information in the measurements. Applying the scaling factor, generally a scale down can have different scaling ratio to be selected, for example 5:1 means 5 mm movement of the device (100) is transformed into 1 mm movement to be controlled.

Subsequent thereof, a velocity determining module (411) can determine a velocity corresponding to the scaled position vector.

Scaled position vector as obtained by the scaling factor application module (410A) and orientation matrix as obtained by the orientation matrix calculation module (408) can be provided to a transformation matrix calculation module (412). The transformation matrix thus derived is indicative of hand motion measurements. The processing device (400) may be enabled to transfer the final hand motion data to other devices (to be controlled) such as a slave manipulator etc.

### EXAMPLES

“The following examples are given by way of illustration of the present invention and therefore should not be construed to limit the scope of the present invention”.

### Example 1

The developed device consists of a combination of planar and spatial mechanical linkages meant to independently sense the motion & pose of a human's hand. This includes motion of shoulder (rotation and swing), arm extension (mimicking elbow joint through a combination of a revolute joint and parallelogram to have better rigidity & stability of the operation), wrist rotation, pitch & yaw of hand and open-close of thumb and index finger. The device is a kinematic chain of rigid bodies connected by suitable joints. The developed device comprises of a base, shoulder swing & rotation, translational, roll, pitch, yaw and gripper linkages each corresponding to human's arm-hand.

A person can manipulate the device by holding it through a handle and graspers on each hand and can move the wrist/arm to perform a skilled/desired action. Schematic of a typical operation of the device is shown in FIG. 2, where a human (surgeon or skilled operator or a patient etc.) holds the invented device through and performs a task. In context to robotic surgery, a slave robot (wired/wireless) can mimic the same action onto a patient undergoing surgery. Each joint of the device is fitted with a sensor (rotary encoder or suitable) to measure the joint angles and ultimately the pose & motion of the human's hand. Pose of human's hand is computed using forward kinematic analysis. The kinematic analysis involves a concept of dummy frames to quantify the position and orientation of the human's fingertip.

The first link is base part (which supports the whole device) and is connected to the second links via a revolute joint. The second link is further joined to the third link via a revolute joint. The joint axes are perpendicular to each other so that two rotary motions in mutually perpendicular direction are measured. This portion of the mechanism corresponds to the shoulder motion of a human. The first link is grounded (or fixedly held) at its proximal end and a distal end is fitted with bearing. The second link is mounted on this bearing and it can be of brass/Aluminum of suitable height. Heights of the first and the second links can be altered according to the height of the operator. The third link is joined with the second link so as to allow shoulder swing motion.

Next a translational linkage in the form of a parallelogram linkage is provided. The parallelogram linkage comprises four links namely seventh link, an eight link, a ninth link and a tenth link connected to each other through revolute joints. The four links of the parallelogram linkage are connected in such a way that when operator holds the handle/gripper, the orientation of parallelogram is parallel to forearm of the operator. The seventh and the ninth links are parallel to forearm of operator and the eighth and the tenth links are parallel to the upper arm. The parallelogram will provide translational motion to the device without much effort applied by operator and will also give rigidity to the device (by reducing the bending moment).

Next to translation, three links namely fourth link, fifth link and sixth link are arranged in a configuration (shown in FIG. 1) to achieve the pitch, yaw and roll motions of the hand. Here, the parallelogram linkage is connected to the three links with revolute joints mimicking the motion/DOF of a human wrist. These joints are mutually perpendicular to each other so that we can get the independent pitch, yaw and roll motions. These three motions (yaw, pitch and roll) and gripper are measured to get the pose. The holding part i.e. handle is provided with graspers for thumb and index finger. All the links have been arranged in such a way so as to increase the workspace by controlling the translational part according to workspace of human hand.

This parallelogram linkage is joined to the fourth link. In particular, the fourth link is connected to the ninth link, but the orientation of the fourth link is in different plane compared to the ninth link.

Sensors (not shown in the FIG. 1) are mounted preferably at each joint to measure the joint positions/orientations. The sensors can be of contact or non-contact type.

The motion is measured by recording the electrical signals obtained from the sensors which are mounted on the respective joints. The motions can be sensed by high resolution position sensors, such as digital encoders, high accuracy potentiometers, hall-effect based position sensors etc, placed on the joints of the device. The output of sensors changes according to the movement by the operator. Measured electrical signal, through transducer, are then converted to measurable physical quantities, by DAQ hardware which acts as interface between the sensor and a processing unit.

Measurement of joint angles is achieved with precision of ±0.02°, leading to maximum theoretical error of ±0.14 mm in position and ±0.06° in orientation. Actual measured errors are ±0.35 mm in position and ±0.09° in orientation respectively.

Different materials can be used for the fabrication of the device according to the requirement viz. Aluminum or its alloys and Acrylonitrile Butadiene Styrene (ABS) polymer etc. Aluminum/ABS can be used because of its high strength and low density, whereas brass/SS304 pins can be used as link connectors because they will carry inertia of links. Aluminum strips of 12 mm wide and 3 mm thick can be used for construction of the mechanism. This physical model of the device has been developed and tested.

### Example 2

The operator held the device through the handle and grasper through the thumb and index finger. The grasper was opened and closed to grasp a pen. This motion (open/close) of the grasper was sensed and the angle as well as motion speed was recorded on the computer (through the electronics).

### Example 3

Starting with the Example 2 above, where a pen was held between the index finger and Thumb. It was seen that there was relative movement between grasper and the forearm i.e. say wrist rotation by 90 degrees clockwise. As the wrist rotated in clockwise direction the angle was displayed on the measurement system.

### Example 4

Starting with the Example 2, 3 above, where a pen was held between the index finger and thumb. Apart from the wrist rotation, the pitch and yaw motions of hand were made by the person and these were recorded and displayed at the measurement system. The pen was moved in such a way to draw a circle on the paper and corresponding motion was recorded and displayed by the measurement system.

### Example 5

Starting with the Examples 2, 3, 4 above, a text “robotic surgery” was written on a paper using the pen as well as a star shape was drawn. It was seen that all the linkages moved corresponding to the natural motion of the hand. It was observed that there was no obstruction or difficulty to the operator while performing above tasks through the device.

### Example 6

When all the activities stated in above Example 2 through Example 5, were integrated and operated, it was observed that the tasks were carried out satisfactorily and motion & pose of human's arm-hand could be recorded in real time.

Measurement of joint angles is achieved with precision of ±0.02°, leading to maximum theoretical error of ±0.14 mm in position and ±0.06° in orientation. Actual measured errors are ±0.35 mm in position and ±0.09° in orientation respectively.

### Advantages

The main advantage of the present invention is that opening/closing of the grasper and other joints of the device are actuated naturally due to human's hand motion. So there is no need of any external motors or continuous power to operate the device. This results in energy saving.

Apart from the above, there are other advantages of the invention. Yet another advantage of the invention is that the device has a modular design so that it can be used in different configurations by retaining the required module only. Yet another advantage of this invention is inertia of each joint of the device is kept minimal such that it minimizes the energy dissipation of human's hand while operating the device. Each joint (actuated during the motion) is supported on bearings for effortless motion transmission from the human to the sensors for subsequent measurements.

Yet another advantage of this invention is that the device has a sensor at the grasper link, so that the operator can grasp/release an object in a virtual environment. Yet another advantage of the present invention is the use of non-contact type encoders, which eliminate the problems of wear and tear and thus improve long term accuracy/repeatability.

Yet another advantage of the present invention is that the low inertia of the device leads to real time motion sensing and thus real-time control of an application based on this data. Yet another advantage of the present invention is that the device is light weight and sits on the sides of human during the operation. Thus there is no loading or obstruction of human hand motion so that natural motion can be recorded without external influence.

Yet another advantage of the present invention is the use of a versatile design so that the device can be used for numerous applications like master-slave robotic surgery, motor-deficient upper limb amputee, rehabilitation & assessment of upper limb amputees, to record the motion of a skilled/professional person like artist, surgeon etc. Yet another advantage of the present invention is the device measures the motion of human's hand through individual links, which are directly coupled to each other through a special pin so that there is no need of gears (at the joints) which can cause backlash and friction etc.

Yet another advantage of the present invention is that the each joint of the device is designed in such a way that there is 1:1 mapping/correspondence to natural joints of human arm. All degrees-of-freedom (positional and orientation) of the motion are decoupled and measured independently. This helps in quantifying the natural motion without any external hindrance.

Yet another advantage of the invention is that almost nil training of the human/operator is required to operate the device. Yet another advantage of the invention is that it has an in built electronic scaling down feature which helps in reducing the vibration effect in measurements. For the applications related to medical robotics (master-slave configuration), this will improve the surgical efficacy. Yet another advantage is that both the upper arm & forearm motions as well as pose can be measured through the single device.

Another advantage of this invention is that all measurements can be made while providing ease, less fatigue to the user of the device. Another advantage of the invention is that both the upper arm & forearm motions as well as pose can be measured through the single mechanism.

Yet another advantage of the invention is the modular design of the device. Because of the modular design, that if any module/link malfunctions, then it can be replaced with the newer one instead of replacing the whole device. Also, because of the modular design, the device can be used in different configurations by retaining the required module only.

Yet another advantage of the invention is that the device provides for sensing of rotation of human body apart from the hand-arm motion, to get complete pose of a human. So this device can be used for measuring the motion of a human performing a task in sitting as well as standing configuration.

Yet another advantage of the invention is that there is no loading or obstruction of human's hand motion so that natural motion can be recorded without external influence. Yet another advantage of the present invention is that the operator can grasp/release an object in a virtual environment because the grasper link can be provided with one or more sensors.

Yet another advantage of the invention is that the device allows for measurement of the motion of human's hand through individual links, which are directly coupled to each other through pins so that there is no need of gears etc. which can cause backlash and friction etc.

Yet another advantage of the invention is that the device allows for opening/closing of the grasper and actuation of other joints of the mechanism naturally due to human's hand motion. So there is no need of any external motors or continuous power to operate the device. This results in energy saving.

Yet another advantage of the invention is that the device incorporates torsion spring in the mechanism to minimize the effect of gravity/inertia so that operator/human does not feel any weight on the hand while manipulation of the device. Yet another advantage of the invention is that the device provides low inertia of the mechanism to achieve real time motion sensing and thus real-time control of an application based on this data.

Still another advantage of the invention is that the device is designed in such a way that there is 1:1 mapping/correspondence to natural joints of human arm. All degrees-of-freedom (positional and orientation) of the motion are decoupled and measured independently.

Still another advantage of the invention is that the control is exhibited using electrical signals and using electronic circuitry. Still another advantage of the invention is that it can implement various types of processing method to compute the pose of hand. Still another advantage of the invention is that almost nil training of the human/operator is required to operate the device.

Still another advantage of the invention is that the device has an in built electronic scaling down feature such that it helps in reducing the vibration effect in measurements. For the applications related to medical robotics (master-slave configuration), this will improve the surgical efficacy. Yet another advantage of the invention is that the energy dissipation of human's hand is minimum while operating the mechanism because inertia of each joint of the mechanism is kept minimal. Yet another advantage of the invention is that the device is inexpensive to manufacture, easy to service and replace parts.

While certain present preferred embodiments of the invention have been illustrated and described herein, it is to be understood that the invention is not limited thereto. Clearly, the invention may be otherwise variously embodied, and practiced within the scope of the above description.

1. A device for measuring pose and/or motion of an operator's hand, the device comprising:
a first link having a proximal end and a distal end, the proximal end being adapted to be fixedly held and the distal end being coupled to a proximal end of a second link so as to allow a rotation motion there between about a first axis;
a distal end of the second link being coupled to a proximal end of a third link so as to allow a rotation motion there between about a second axis, the second axis being substantially perpendicular to the first axis;
a parallelogram linkage defining a first edge and a diametrically opposite second edge, the first edge of the parallelogram linkage being coupled to a distal end of the third link;
the second edge of the parallelogram linkage being coupled to a proximal end of a fourth link;
a distal end of the fourth link being coupled to a proximal end of a fifth link, a distal end thereof being further coupled to a proximal end of a sixth link; and
a grasper being operably coupled to a distal end of the sixth link;
wherein each of said links and said parallelogram-linkage is implemented by a revolute-joint and at least one-sensor fitted to each revolute joint for measuring an orientation thereof.

2. The device as claimed in claim 1, wherein the parallelogram linkage comprises a seventh link, an eight link, a ninth link and a tenth link connected to each other through revolute joints such that the seventh link and the ninth link are parallel to each other and in an operating state are located parallel to a forearm of the operator and the eighth link and the tenth link are parallel to each other and in an operating state are located parallel to an upper arm of the operator.

3. The device as claimed in claim 1, wherein the fourth link, the fifth ink and the sixth link in operation are adapted to mimic an operator's wrist movement.

4. The device as claimed in claim 1, wherein the grasper defines a first element adapted to contact a thumb of an operator and a second element adapted to be contact at least one of the remaining fingers of the operator.

5. The device as claimed in claim 1, further comprising a processing device.

6. The device as claimed in claim 5, wherein the processing device is operably coupled to at least one sensor via one or more data acquisition hardware units.

7. The device as claimed in claim 6, wherein the at least one sensor is selected from a group comprising of:
a sensor disposed at a location of coupling of the first link and the second link (joint between the first link and the second link);
a sensor disposed at a location of coupling of the second link and the third link (joint between the second link and the third link);
a sensor disposed at a location of coupling of the third link and the parallelogram linkage (joint between the third link and the parallelogram linkage);
a sensor disposed at a location of coupling of the parallelogram linkage and the fourth link (joint between the parallelogram linkage and the fourth link);
a sensor disposed at a location of coupling of the fourth link and the fifth link (joint between the fourth link and the fifth link);
a sensor disposed at a location of coupling of fifth link and the sixth link (joint between the fifth link and the sixth link);
a sensor disposed at a location of coupling of the sixth link and the grasper (joint between the sixth link and the grasper);
a sensor disposed at a location of coupling of the seventh link and the eighth link (joint between the seventh link and the eighth link);
a sensor disposed at a location of coupling of the eighth link and the ninth link (joint between the eighth link and the ninth link);
a sensor disposed at a location of coupling of the ninth link and the tenth link (joint between the ninth link and the tenth link);
a sensor disposed at a location of coupling of the tenth link and the seventh link (joint between the tenth link and the seventh link);
a sensor disposed at about the first element; and
a sensor disposed at about the second element.

8. The device as claimed in claim 5, wherein the processing device is adapted to:
Obtain input from the at least one sensor via the at least one data acquisition hardware;
Determine a pose of the hand of the operator based on the sensor's input and obtain information pertaining to a relationship between individual joint and the corresponding link;
Determine angular position of a joint corresponding to the sensor's input;
Compute a rate of change of each angular position;
Calculate joint angles or joint spaces on basis of rate of change of angular positions and information pertaining to the device's limited range of motion;
Transform the joint angles or joint spaces into corresponding position in Cartesian space on the basis of the information pertaining to the relationship between individual joint and the corresponding link;
Obtain position vector and orientation matrix;
Detect a change in position vector;
Apply a scaling ratio to obtained scaled position vectors;
Derive a velocity on basis of the scaled position vectors thus obtained; and
Derive a transformation matrix indicative of hand motion measurements on basis of the orientation matrix and the scaled position vectors thus obtained.

9. The device as claimed in claim 8, wherein the step of determining a pose of the hand of the operator includes processing sensor inputs pertaining to yaw motion, pitch motion and roll motion of the fourth link, the fifth link, the sixth link and the grasper.

10. The device as claimed in claim 1, further comprising at least one torsion spring for minimizing effect of gravity/inertia on human's hand while operating the device.

11. The device as claimed in claim 1, wherein the device has at least one of:
the first link having an adjustable length;
the second link having an adjustable length;
the third link having an adjustable length;
the parallelogram linkage having an adjustable size;
the fourth link having an adjustable length;
the fifth link having an adjustable length; and
the sixth link having an adjustable length.
